(19) United States
(12) Patent Application Publication
(10) Pub. No.: US 2025/0332181 A1
(43) Pub. Date: Oct. 30, 2025

(54) ANTIBIOTIC METHODS AND COMPOSITIONS

(71) Applicant: AI BIO DISCOVERY LLC, Bronxville, NY (US)

(72) Inventors: Manfred Philipp, Bronxville, NY (US); Purushotam Prasai, Kathmandu (NP)

(73) Assignee: AI BIO DISCOVERY LLC, Bronxville, NY (US)

(21) Appl. No.: 19/195,203

(22) Filed: Apr. 30, 2025

Related U.S. Application Data
(60) Provisional application No. 63/640,835, filed on Apr. 30, 2024.

(51) Int. Cl. A61K 31/69; A61K 45/06; A61P 31/04

(52) U.S. Cl. CPC A61K 31/69; A61K 45/06; A61P 31/04

(57) ABSTRACT

An antibiotic therapeutic composition or method of treatment includes administering a beta-lactamase inhibitor in combination with a beta-lactam antibiotic. The beta-lactamase inhibitor can be a boronic-acid, a boronic-ester beta-lactamase inhibitor, carboxyl-group beta-lactamase inhibitors, halogen-terminated heterocyclic group beta-lactamase inhibitors, or any pharmaceutically acceptable salts thereof. In combination, the beta-lactamase inhibitors and beta-lactam antibiotics can inhibit the growth of bacteria that are resistant to the beta-lactam antibiotics alone. According to additional embodiments, the effectiveness of various combinations of compounds including various antibiotics and compounds selected from the group consisting of 5-Carboxythiophene-2-boronic acid pinacol ester, 5-Carboxy-2-fluorophenylboronic Acid, 3-Amino-5-carboxylphenylboronic acid, 5-Carboxy-2-chlorophenylboronic acid, 3-Carboxy-2-fluorophenylboronic acid, 5-dehydroxyboryl-2-thiophenecarboxylic acid, and 5-Borono-2-chlorobenzoic acid is demonstrated. Particularly, when combined with certain antibiotics, each of these compounds demonstrates enhanced effectiveness against bacteria and bacterial infections even where the bacteria are known to be resistant to the beta-lactam antibiotics.

### CROSS REFERENCE TO RELATED APPLICATION

This application is a U.S. Non-provisional patent application of U.S. Provisional Patent Application No. 63/567,982 entitled “Retractable Seat Cover System,” filed Mar. 21, 2024, which is herein incorporated by reference in its entirety for all purposes.

### FIELD OF THE INVENTION

The present invention relates to a seat protector, and more particularly to a retractable vehicle seat cover which can be conveniently attached, removed, or stored. The cover may be deployed from bottom-to-top or from top-to-bottom.

### BACKGROUND

Beta-lactamases (β-lactamases) are the primary cause of bacterial resistance to beta-lactam (β-lactam) antibiotics. A traditional approach to improving the efficacy of beta-lactam antibiotics has been to modify natural beta-lactam antibiotics by adding various side chains. It is generally believed that beta-lactam antibiotics have a limited future given increased resistance is demonstrated by many strains of bacteria. Because of the rise of resistant bacterial strains to traditional beta-lactam antibiotics, there is a need for novel antibiotic treatment methods and compositions.

### SUMMARY

Antibacterial treatment compositions and methods are provided herein that include a beta-lactam antibiotic and a beta-lactamase inhibitor. In some cases, the beta-lactamase inhibitor can be a boronic-acid beta-lactamase inhibitor or a pharmaceutically acceptable salt thereof. In some cases, the beta-lactamase inhibitor can be a boronic-ester beta-lactamase inhibitors or a pharmaceutically acceptable salt thereof. In some cases, the beta-lactamase inhibitor includes a boronic-acid group or an ester thereof. In some cases, the beta-lactamase inhibitor includes a carboxyl group. In some cases, the beta-lactamase inhibitor includes a cyclic group. In some cases, the beta-lactamase inhibitor is a heterocyclic compound. In some cases, the beta-lactamase inhibitor includes one or more atoms selected from the group consisting of S, Se, B, and O as members of a heterocyclic ring. In some cases, the beta-lactamase inhibitor comprises a ring structure comprising 5 atoms. In some cases, the beta-lactamase inhibitor includes a thiophene ring. In some cases, the beta-lactamase inhibitor includes one or more halogen groups (e.g., F). In some cases, a halogen group can be bound to a ring structure (e.g., a heterocyclic ring, such as a thiophene ring). In some cases, the beta-lactamase inhibitor includes a single boronic-acid group. In some cases, the beta-lactamase inhibitor can include two or more boronic-acid groups. In some cases, the beta-lactamase inhibitor includes a single boronic-ester group. In some cases, the beta-lactamase inhibitor can include two or more boronic-ester groups. For example, in some cases, the beta-lactamase inhibitor can be 2-carboxythiophene-5-boronic Acid. For example, in some cases, the beta-lactamase inhibitor can be 5-carboxythiophene-2-boronic acid pinacol ester.

According to some embodiments of the present disclosure, an antibacterial composition includes a beta-lactam antibiotic and a beta-lactamase inhibitor. In some cases, the molar ratio of the beta-lactamase inhibitor to the beta-lactam antibiotic can be greater than 1. In some cases, the molar ratio of the beta-lactamase inhibitor to the beta-lactam antibiotic can be greater than 2, 3, 5, 8, 10, 25, or 100. In some cases, the weight ratio of the beta-lactamase inhibitor to the beta-lactam antibiotic can be between 0.2-100, between 0.3 to 50, between 0.5 to 25, between 0.8 to 10, or between 1 and 5. In some cases, the composition is in the form of a pill. In some cases, a pill including the composition intended to treat an animal can include between 1 and 10 mg per kg (of animal weight) of the beta-lactam antibiotic and between 0.5 and 100 mg per kg (of animal weight) of the beta-lactamase inhibitor. In addition to introducing the composition to the user by way of a pill, the composition may be administered intravenously or by way of a patch or subcutaneous injections.

According to some embodiments of the present disclosure, a method of treating a bacterial infection can include administering both a beta-lactam antibiotic and a beta-lactamase inhibitor to treat the bacterial infection. In some embodiments, the bacteria of the bacterial infection can be resistant to the beta-lactam antibiotic alone. In some cases, the beta-lactamase inhibitor alone (without the beta-lactam antibiotic) does not provide any significant anti-microbial activity against the bacterial infection. In some cases, methods provided herein include administering the beta-lactam antibiotic and the beta-lactamase inhibitor together as a single composition or administering the beta-lactam antibiotic and the beta-lactamase inhibitor separately. For example, in some cases, a bacterial infection can include bacteria that is penicillin-resistant bacteria, and the bacterial infection can be treated with penicillin and the beta-lactamase inhibitors described herein (e.g., 2-carboxythiophene-5-boronic acid and/or 5-carboxylthiophene-2-boronic acid pinacol ester).

According to the present invention, Applicant has made additional advances in establishing the effectiveness of various combinations of compounds Applicant developed with known antibiotics. These compounds include 5-(Dihydroxyboryl)-2-thiophenecarboxylic acid, 5-Carboxythiophene-2-boronic acid pinacol ester, 5-Carboxy-2-fluorophenylboronic acid, 3-Amino-5-carboxylphenylboronic acid, 5-Carboxy-2-chlorophenylboronic acid, 3-Carboxy-2-fluorophenylboronic acid, and 5-Borono-2-chlorobenzoic acid. When combined with certain antibiotics and as set forth below, each of these compounds demonstrates enhanced effectiveness against bacteria and bacterial infections even where the bacteria are known to be resistant to the beta-lactam antibiotics.

The various embodiments and examples described in the summary and this document are provided not to limit or define the disclosure or the scope of the claims.

### BRIEF DESCRIPTION OF THE FIGURES

FIG. 1A depicts the chemical structure of an exemplary beta-lactamase inhibitor, namely 2-carboxythiophene-5-boronic acid selected for use according to the present disclosure;

FIG. 1B depicts an ester of the beta-lactamase inhibitor of FIG. 1A, namely 5-carboxylthiophene-2-boronic acid pinacol ester selected for use according to the present disclosure;

FIG. 1C is another example of a beta-lactamase inhibitor, namely 4-boronothiophene-2-carboxylic acid selected for use according to the present disclosure;

FIGS. 2A-2N depict additional exemplary beta-lactamase inhibitors selected for use according to the present disclosure;

FIG. 3A depicts the chemical structure of ceftazidime, a beta-lactam antibiotic selected for use according to the present disclosure;

FIG. 3B depicts the chemical structure of penicillin G (benzyl penicillin), a beta-lactam antibiotic selected for use according to the present disclosure;

FIG. 3C depicts the chemical structure of meropenem, a beta-lactam antibiotic selected for use according to the present disclosure;

FIG. 3D depicts the chemical structure of cefoxitin, a beta-lactam antibiotic selected for use according to the present disclosure;

FIG. 4A is a photograph of disk diffusion results using ceftazidime on Klebsiella pneumoniae;

FIG. 4B is a line drawing of the agar plate illustrated in FIG. 4A;

FIG. 5A is a photograph of disk diffusion results using ceftazidime on Klebsiella pneumoniae;

FIG. 5B is a line drawing of the agar plate illustrated in FIG. 5A;

FIG. 6A is a photograph of disk diffusion results using penicillin G on methicillin-resistant coagulase-negative Staphylococcus sps. (MRCoNS);

FIG. 6B is a line drawing of the agar plate illustrated in FIG. 6A;

FIG. 7A is a photograph of disk diffusion results using ceftazidime on Klebsiella pneumoniae;

FIG. 7B is a line drawing of the agar plate illustrated in FIG. 7A;

FIG. 8A is a photograph of disk diffusion results using meropenem and ceftazidime on Klebsiella pneumoniae;

FIG. 8B is a line drawing of the agar plate illustrated in FIG. 8A;

FIG. 9A is a photograph of disk diffusion results using cefoxitin and penicillin G on methicillin-resistant coagulase-negative Staphylococcus sps. (MRCoNS);

FIG. 9B is a line drawing of the agar plate illustrated in FIG. 9A;

FIG. 10 is a chart summarizing test results based upon the use of a combination of 5-(Dihydroxyboryl)-2-thiophenecarboxylic acid with various antibiotics;

FIG. 11 is a chart summarizing test results based upon the use of a combination of 5-Carboxythiophene-2-boronic acid pinacol ester with various antibiotics;

FIG. 12 is a chart summarizing test results based upon the use of a combination of 5-Carboxy-2-fluorophenylboronic Acid with various antibiotics;

FIG. 13 is a chart summarizing test results based upon the use of a combination of 3-Amino-5-carboxylphenylboronic acid with various antibiotics;

FIG. 14 is a chart summarizing test results based upon the use of a combination of 5-Carboxy-2-chlorophenylboronic acid with various antibiotics;

FIG. 15 is a chart summarizing test results based upon the use of a combination of 3-Carboxy-2-fluorophenylboronic acid with various antibiotics; and

FIG. 16 is a chart summarizing test results based upon the use of a combination of 5-Borono-2-chlorobenzoic acid with various antibiotics.

FIG. 17 is a chart summarizing test results based upon the use of a combination of 2,3,5-Trifluorophenylboronic acid with various antibiotics.

FIG. 18 is a chart summarizing test results based upon the use of a combination of Benzoic acid, 3-borono-5-nitro with various antibiotics.

FIG. 19 is a chart summarizing test results based upon the use of a combination of 5-Borono-2-fluorobenzoic acid with various antibiotics.

FIG. 20 is a chart summarizing test results based upon the use of a combination of Phenylacetic acid-3-boronic acid pinacol ester with various antibiotics.

FIG. 21 depicts the chemical structure of an exemplary boronic acid having a n-butyl group on the boron atom;

FIG. 22 depicts the chemical structure of the boronic acid of FIG. 21 in a cyclic form; and

FIG. 23 depicts the chemical structure of a cyclic boronic acid ester.

### DETAILED DESCRIPTION

Antibacterial treatment compositions and methods include the combination of beta-lactamase inhibitors with beta-lactam antibiotics to treat bacterial infections that include bacteria that are resistant to the beta-lactam antibiotics.

The disclosed treatment compounds work most effectively when the bacteria are growing in a log phase according to Applicants' investigations. Applicants also found that their solutions to antibiotic-resistant treatments disclosed herein are not likely anticipated because of the type of bacterial strains commonly available from, for example, the ATCC. Conversely, it is known that clinical isolates from hospital patients having bacterial resistance to almost all or all available antibiotics (MDR [multiple drug resistant] and XDR [extensively drug-resistant] strains) reveal that such resistance is increasing particularly in certain regions such as southeast Asia and sub-Sahara Africa. The inventors of the present composition found that their agents were only active to antibiotic resistant strains of the bacteria used by them. More directly, when the inventors first applied their composition against standard ATCC strains, they were found to have no effect at all.

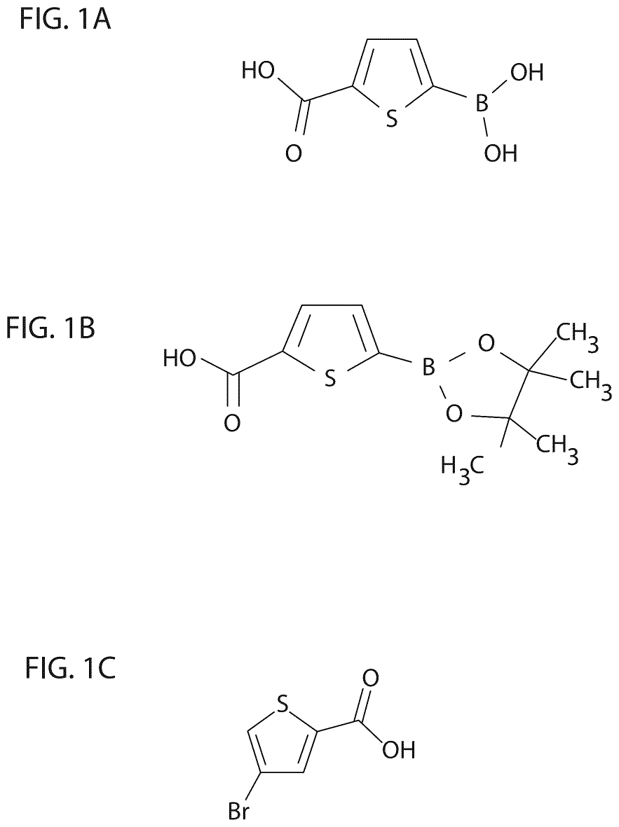

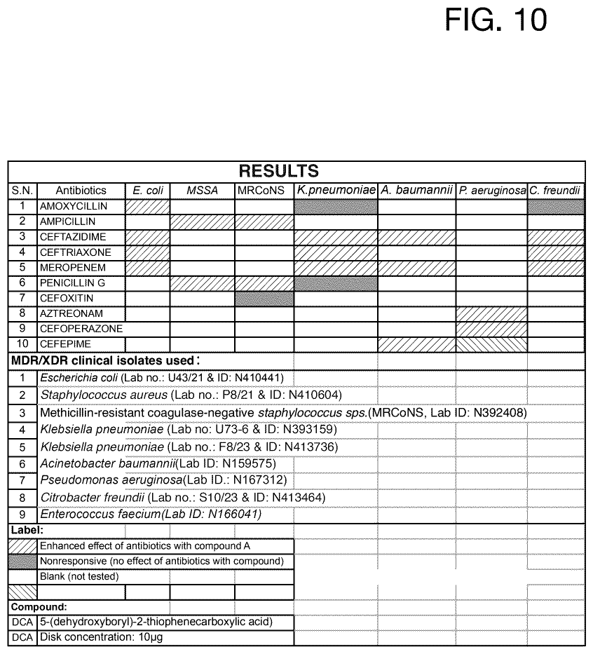

Applicants undertook two rounds of studies. The results of the first round are illustrated in FIGS. 1A-9B and discussed in the related text. The results of the second round of studies are illustrated in FIGS. 10-23 and discussed in the related text. When agar plating was required, both rounds of Applicants' studies were undertaken using similar disk diffusion methods on bacteria grown on agar substrates in Petri dishes. These methods conform to those prescribed by the Clinical & Laboratory Standards Institute (https://clsi.org).

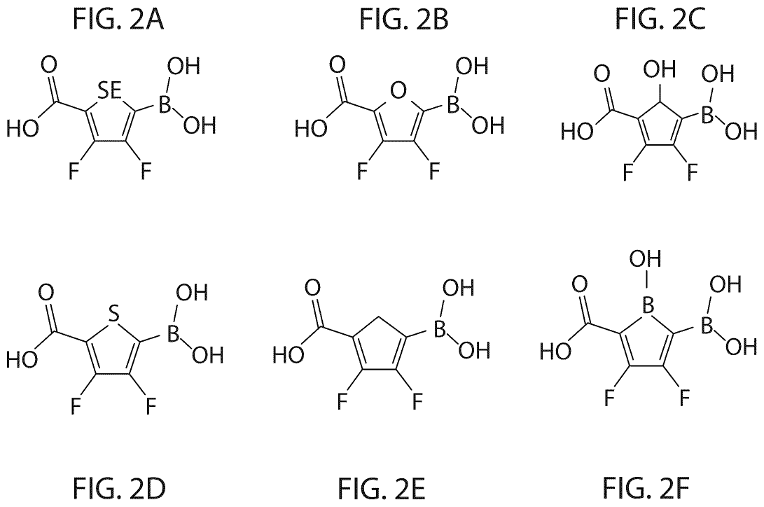

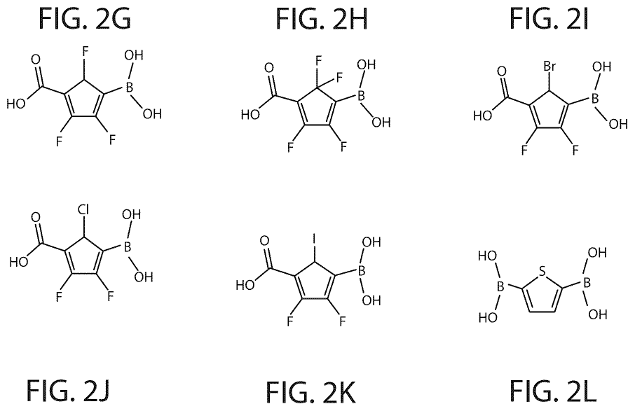

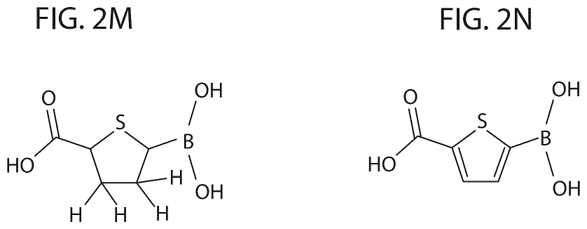

FIG. 1A is an example beta-lactamase inhibitor, 2-carboxythiophene-5-boronic Acid, that can be used in combination with beta-lactam antibiotics to prevent bacterial growth and/or treat bacterial infections. FIG. 1B is another example beta-lactamase inhibitor, 5-carboxylthiophene-2-boronic acid pinacol ester, which can be used in combination with beta-lactam antibiotics. FIG. 1C is another example beta-lactamase inhibitor, 4-boronothiophene-2-carboxylic acid. FIGS. 2A-2O depict alternative beta-lactamase inhibitors suitable for combination with beta-lactam antibiotics. In some cases, the beta-lactamase inhibitor can have the following structure:

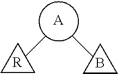

Wherein A is a cyclic group, B is a boronic acid, boronic ester group, or a halogen, and R is a carboxyl group, a sulfonic acid group, a phosphonic acid group, a boronic acid group or a boronic ester group.

In some cases, the A cyclic group can be a heterocyclic group. In some cases, the A cyclic group can include carbon atoms and one or more atoms selected from S, Se, B, and O. In some cases, the A cyclic group includes a ring structure including 5 atoms. In some cases, as shown in FIGS. 1A, 1B, 1C, 2D, 2L, 2N, and 2O, the A cyclic group can be a thiophene ring. In some cases, such as shown in FIG. 2M, the A cyclic group can be a tetrahydrothiophene ring. In some cases, such as shown in FIG. 2A, the A cyclic group can be a selenophene. In some cases, such as shown in FIG. 2B, the A cyclic group can be a furan ring. In some cases, such as shown in FIG. 2F, the A cyclic group can include a boronic acid. In some cases, such as shown in FIGS. 2C and 2F, the A cyclic group can include a terminal hydroxyl group. In some cases, such as shown in FIG. 2F, the A cyclic group can include a borinic acid. In some cases, the A cyclic group can be bound to terminal hydrogens, terminal deuterium atoms, and/or terminal tritium atoms.

In some cases, group B is a boronic acid group or a boronic acid group that is esterified to any alcohol or any diol. In some cases, the beta-lactamase inhibitor includes a single boronic-acid group. In some cases, such as shown in FIG. 2L, the beta-lactamase inhibitor can include two or more boronic-acid groups. In some cases, the beta-lactamase inhibitor includes a single boronic-ester group. In some cases, the beta-lactamase inhibitor can include two or more boronic-ester groups.

In some cases, such as shown in FIGS. 1A, 1B, 2A-2K, and 2M-2N, the R group can be a carboxyl group. In some cases, such as shown in FIG. 2L, the R group can be a boronic acid. In some cases, the R group can be a boronic ester. In some cases, the R group can be the carboxyl group which can be replaced by a sulfonic acid group or a phosphonic acid group.

For example, in some cases, the beta-lactamase inhibitor can be 2-carboxythiophene 5-boronic acid, such as shown in FIG. 1A. For example, in some cases, the beta-lactamase inhibitor can be 5-carboxylthiophene-2-boronic acid pinacol ester, such as shown in FIG. 1B.

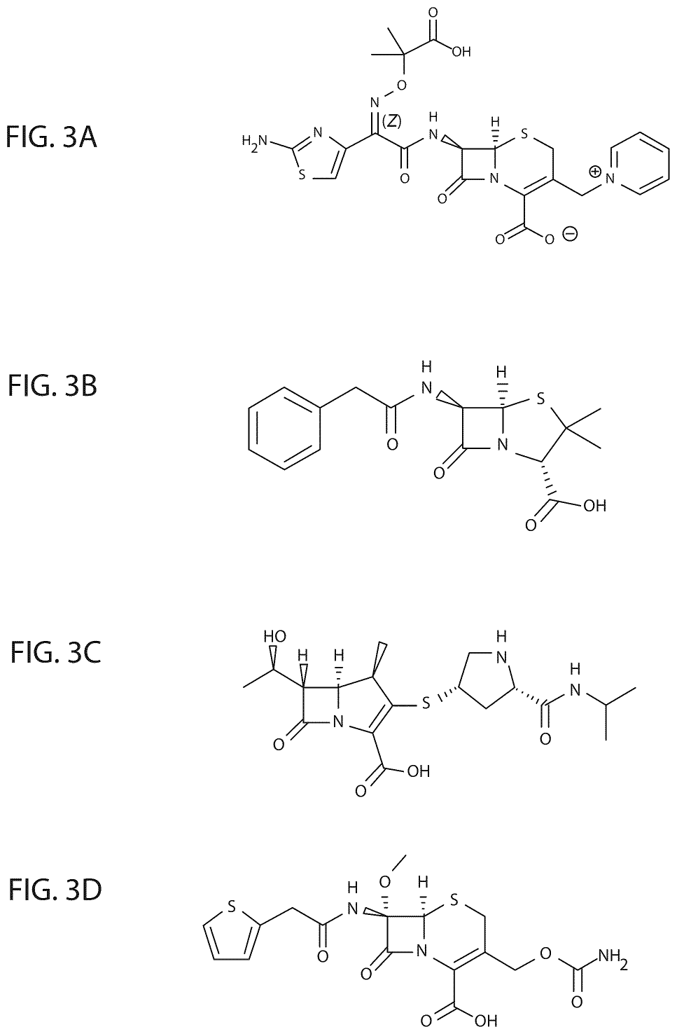

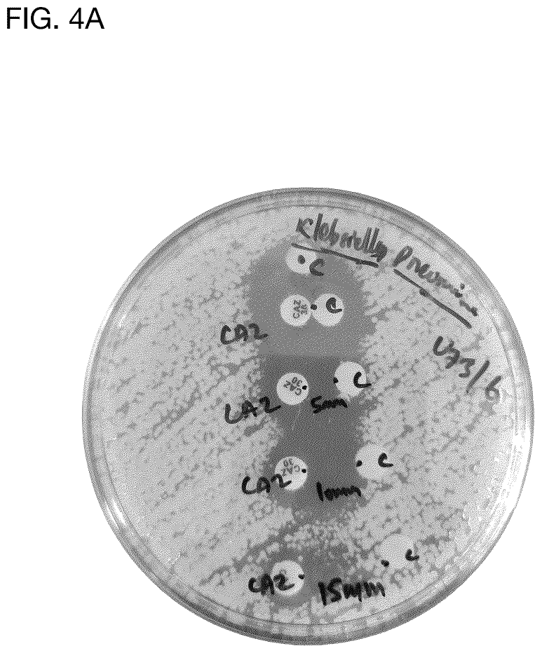

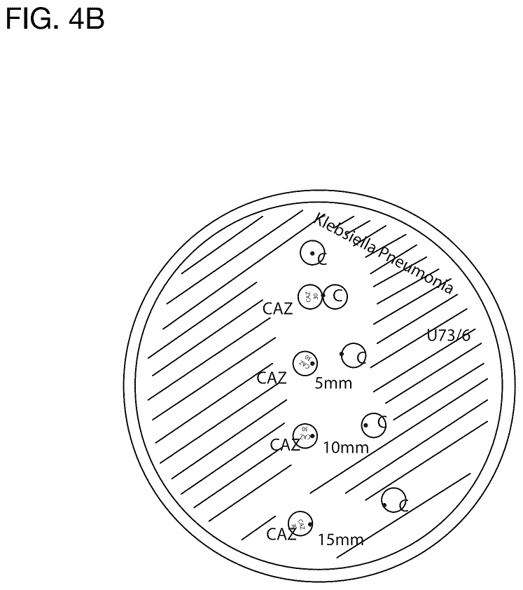

Disk Diffusion tests with beta-lactam antibiotics with and without the beta-lactamase inhibitor of FIG. 1A. These disk diffusion tests are shown in FIGS. 4A-9B. Closely spaced disks disclose possible synergistic or inhibitory effects between the antibiotics and the compound of FIG. 1A (labeled in FIGS. 4A-9B as Compound C). The distances between disks show the effectiveness of the possible synergy. Similar results were achieved with the compound of FIG. 1B. Each disk contains either the compound of FIG. 1A or an antibiotic (one of FIGS. 3A-3D), or the compound of FIG. 1B and an antibiotic (one of FIGS. 3A-3D). When a disk with the compound of FIG. 1A is in proximity to a disk with an antibiotic both agents diffuse into the bacterial growth medium, allowing us to observe the area where both agents are present at the same time. These disk diffusion tests were evaluated with the following:

FIGS. 4A and 4B—Ceftazidime and Klebsiella pneumoniae, with and without proximity to a disk with the compound of FIG. 1A (labeled as Compound).

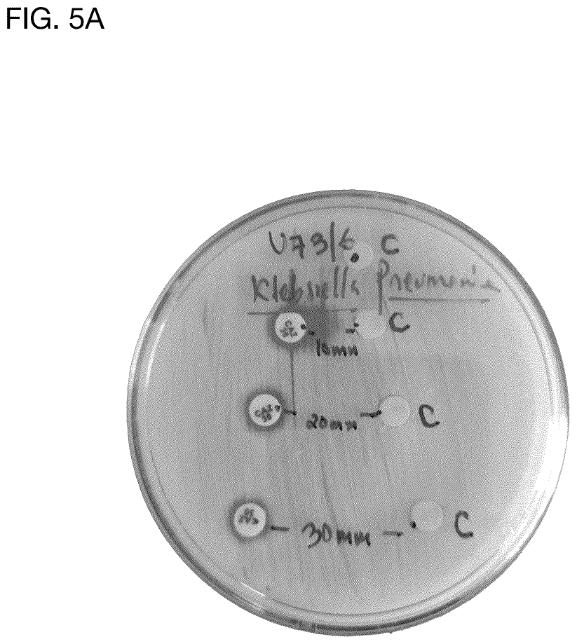

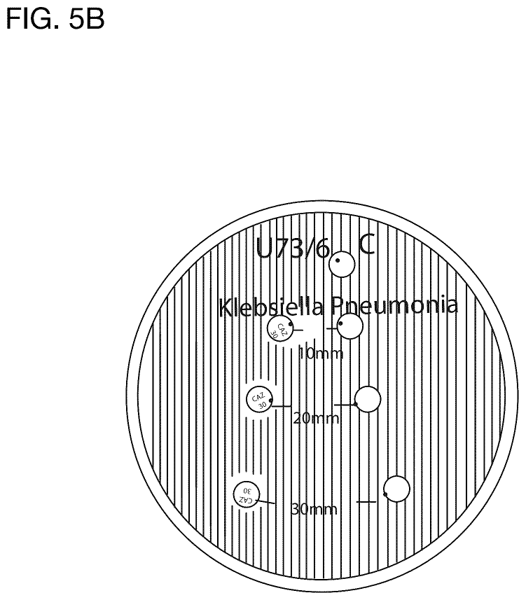

FIGS. 5A and 5B—Ceftazidime on Klebsiella pneumoniae with and without proximity to a disk containing the compound of FIG. 1A (labeled as Compound).

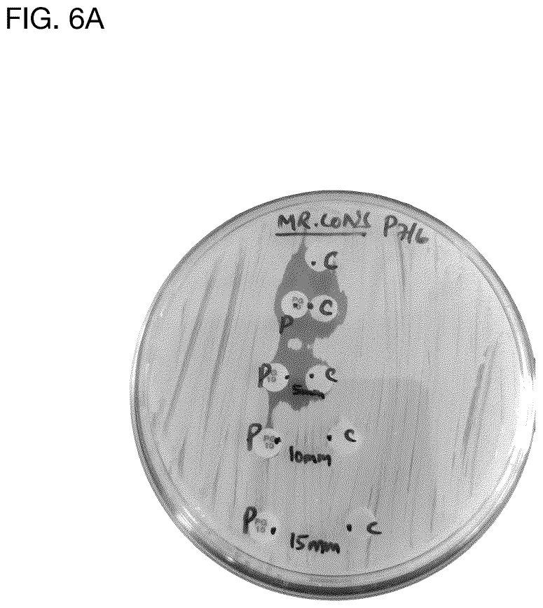

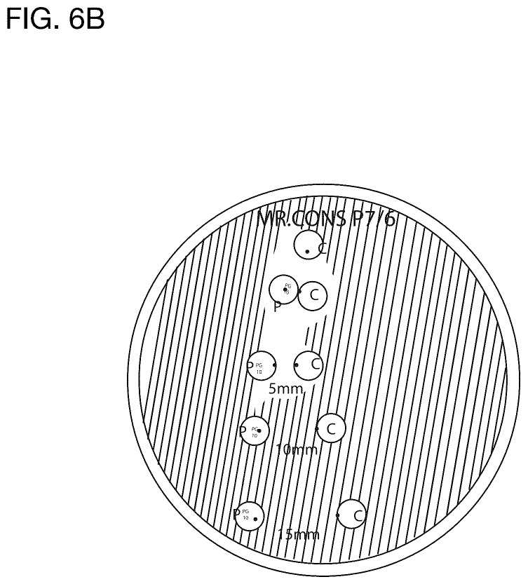

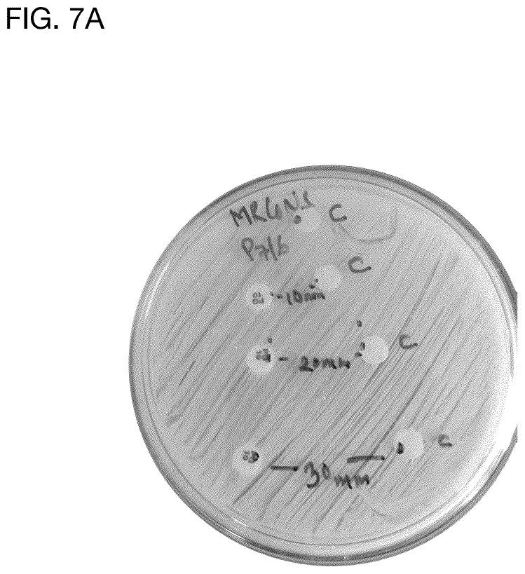

FIGS. 6A and 6B—Penicillin G on methicillin-resistant coagulase-negative Staphylococcus sps. (MRCoNS), with and without proximity to a disk containing the compound of FIG. 1A (labeled as Compound) on the same disk.

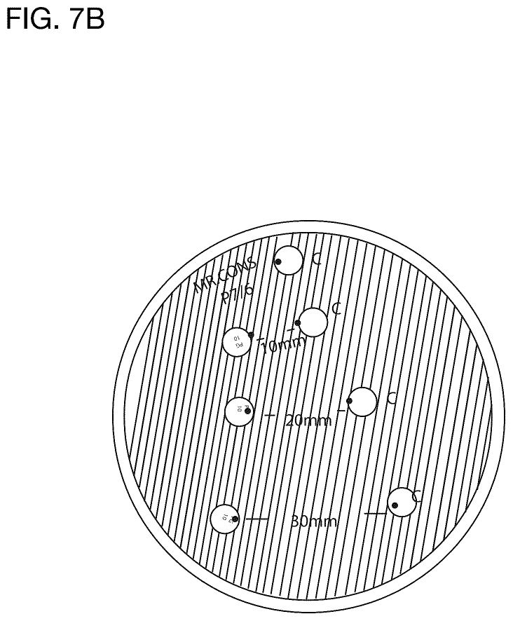

FIGS. 7A and 7B—Penicillin G on methicillin-resistant coagulase-negative Staphylococcus sps. (MRCoNS), with and without proximity to a disk containing the compound of FIG. 1A (labeled as Compound) on the same disk.

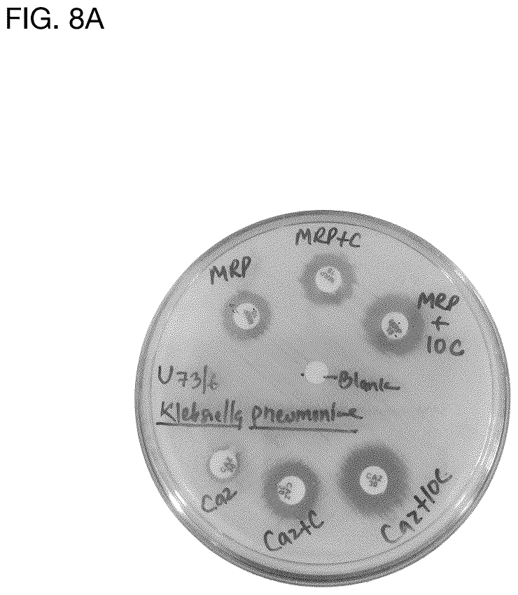

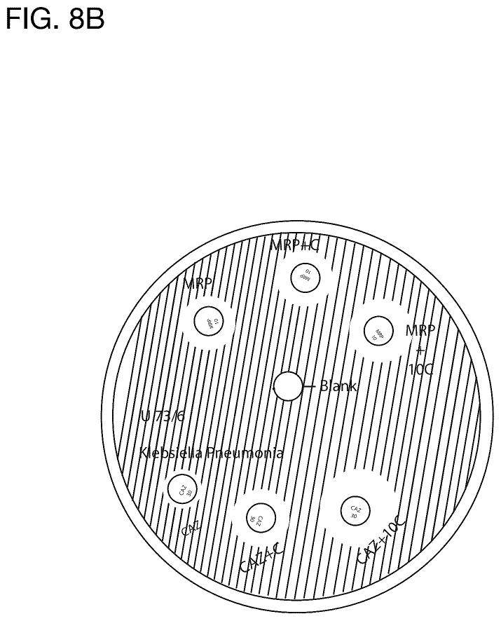

FIGS. 8A and 8B—Meropenem and ceftazidime on Klebsiella pneumoniae with and without a disk containing the compound of FIG. 1A (labeled as Compound).

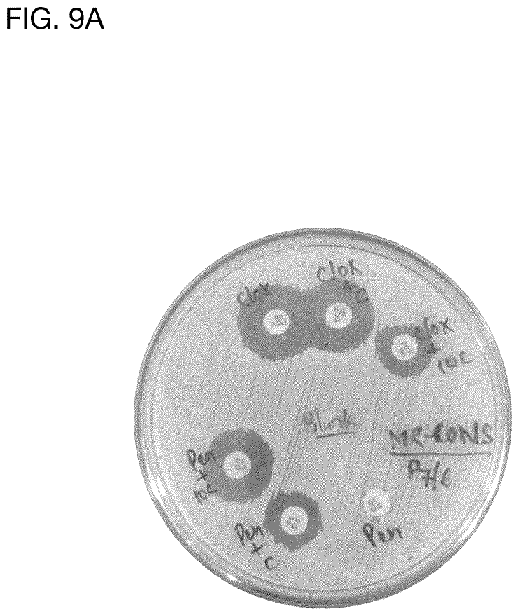

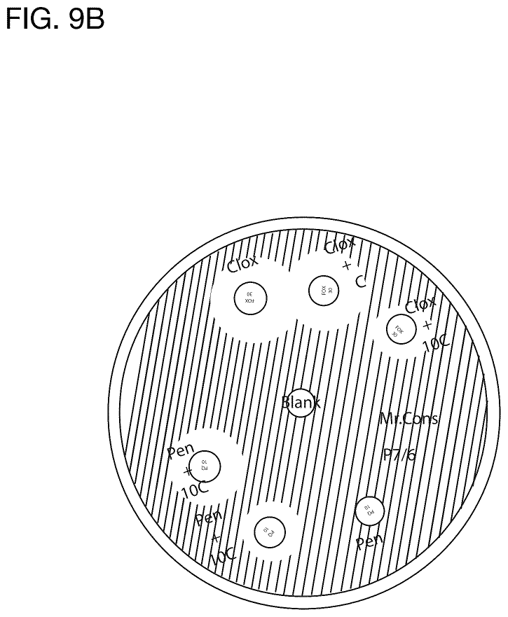

FIGS. 9A and 9B—Cefoxitin and penicillin G on methicillin-resistant coagulase-negative Staphylococcus sps. (MRCoNS), with and without a disk containing the compound of FIG. 1A (labeled as Compound).

As shown in FIGS. 4A, 4B, 5A and 5B, the compound of FIG. 1A (labeled and referenced as Compound C) very strongly enhances the action of ceftazidime (labeled as CAZ) on Klebsiella pneumoniae. The ceftazidime deposits included 30 μg and the Compound C deposits included 15 μg. Ceftazidime itself shows very little antibacterial activity in this test. The inventors' lead compound alone shows no antibacterial activity. Ceftazidime (labeled as CAZ) shows strong antibacterial activity when in the presence and proximity to Compound C, which has no activity on its own.

As shown in FIGS. 6A, 6B, 7A, and 7B, the compound of FIG. 1A (Compound C) strongly enables the action of penicillin G on methicillin-resistant coagulase-negative Staphylococcus sps. (MRCoNS). Both penicillin G and Compound C by themselves show no antibacterial activity in this test. P=Penicillin G (10 μg); C=Compound C (15 μg).

As shown in FIGS. 8A and 8B, Compound C (the compound of FIG. 1A) only weakly enhances the action of merophenem (MRP), which is active by itself, while Compound C strongly enhances the activity of ceftazidime (CAZ), which itself is inactive. MRP=Meropenem (10 μg) (by itself IS active). MRP+C=Meropenem+Compound C (15 μg). This level of C has essentially no effect on the activity of meropenem. MRP+10 C=Meropenem+Compound C (150 μg). A higher level of Compound C only weakly enhances the action of meropenem. Blank=Control. Caz=Ceftazidime (30 μg) (by itself is inactive). Caz+C=Ceftazidime+Compound C (15 μg). Compound C strongly enhances the activity of ceftazidime. Caz+10 C=Ceftazidime+Compound C (150 μg)

As shown in FIGS. 9A and 9B, Compound C (the compound of FIG. 1A) does not enhance cefoxitin activity as normal levels and inhibits cefoxitin antibiotic activity had higher levels, while cefoxitin is active on its own, but activates penicillin G (which is inactive on its own). Clox=Cefoxitin (30 μg)—This antibiotic is active on its own. Clox+C=Cefoxitin (30 μg)+Compound C (15 μg)—Compound C does not enhance cefoxitin activity. Clox+10 C=Cefoxitin (30 μg)+Compound (150 μg)—Compound C at this higher level inhibits cefoxitin antibiotic activity. Blank—Control. Pen=Penicillin G (10 μg)—Penicillin G has no activity by itself. Pen+C=Penicillin G (10 μg)+Compound C (15 μg)—Compound C activates the antibiotic action of penicillin G. Pen+10 C=Penicillin G (10 μg)+Compound C (150 μg)—Higher levels of Compound C further activate penicillin G.

The results shown demonstrate that the compound of FIG. 1A (Compound C) can strongly activate selected members of the penicillin and cephalosporin families of beta-lactam antibiotics. These are antibiotics that have lost their ability to inhibit bacterial growth due to the development of bacterial resistance to beta-lactams. Compound C does not positively improve the activity of beta-lactam antibiotics (penicillins and cephalosporins) that are by themselves active in halting bacterial growth. Thus, Compound C and its possible congeners are possible adjuncts to the use of beta-lactam antibiotics that have lost their former activity due to the development of bacterial resistance.

In addition to the study results presented in FIGS. 1A-9B, Applicants found that other combinations provide unexpected beneficial results that extend into new treatment possibilities against bacterial strains that are highly pathogenic to human beings. Many of these strains of the bacteria used in the inventors' work have become resistant to many currently used antibiotics. These bacterial strains cause serious human diseases and often lead to death. More particularly, the strains of the bacteria used in Applicants' work are completely resistant to many of the antibiotics used with those bacteria For instance, Applicants found the strain of Staphylococcus aureus to be completely resistant to the action of both amoxicillin and penicillin G used either separately or in combination.

More particularly, the inventors made additional advances in establishing the effectiveness of various combinations of compounds against a broad variety of previously untreatable disease conditions through combinations with antibiotics which, by themselves, have only limited if any impact on these disease conditions. These compounds include (1) 5-(Dihydroxyboryl)-2-thiophenecarboxylic acid, (2) 5-Carboxythiophene-2-boronic acid pinacol ester, (3) 5-Carboxy-2-fluorophenylboronic Acid, (4) 3-Amino-5-carboxylphenylboronic acid, (5) 5-Carboxy-2-chlorophenylboronic acid, (6) 3-Carboxy-2-fluorophenylboronic acid, and (7) 5-Borono-2-chlorobenzoic acid. When combined with certain antibiotics and as set forth below, each of these compounds demonstrates enhanced effectiveness against bacteria and bacterial infections even where the bacteria are known to be resistant to the beta-lactam antibiotics. None of the seven agents, by themselves, show antibacterial activity.

As noted above, Applicants' studies were undertaken using similar disk diffusion methods on bacteria grown on agar substrates in Petri dishes. These methods conform to those prescribed by the Clinical & Laboratory Standards Institute (https://clsi.org).

During the course of their studies, the inventors achieved success using their proprietary composition against various bacteria with positive results. The tested bacteria include:

Staphylococcus aureus: The disclosed agents restored the antibacterial action of penicillin G and ampicillin against this group of bacteria.

Methicillin-resistant coagulase-negative staphylococci (MRCoNS): The disclosed agents restored the antibacterial action of penicillin G and ampicillin against this group of bacteria.

Klebsiella pneumoniae: The disclosed agents restored the antibacterial action of ceftazidime, ceftriaxone, meropenem, and cefepime against this group of bacteria.

Acinetobacter baumannii: The disclosed agents restored the antibacterial action of ceftazidime, meropenem, and cefepime against this group of bacteria.

Pseudomonas aeruginosa: The disclosed agents restored the antibacterial action of meropenem, cefepime, aztreonam, ceftazidime, and piperacillin against this group of bacteria.

Citrobacter freundii: The disclosed agents restored the antibacterial action of ceftazidime, ceftriaxone, and meropenem against this group of bacteria.

Escherichia coli: The disclosed agents restored the antibacterial action of amoxicillin, ceftazidime, ceftriaxone, and meropenem against this group of bacteria.

Applicants' studies were directed to combinations of antibiotics and bacterial strains where the antibiotics were completely inactive. The reported results are so limited. Applicants further discovered that while two of their agents could not activate amoxicillin towards Citrobacter freundii, each of the bacterial strains studied could be countered by at least two of their agents in combination with at least two of the antibiotics.

Applicants additionally found that the dosage level responsible for restoration of antibiotic activity was in all but one case at or lower than the dosage needed for the action of these antibiotics in cases where the bacterial strains have not acquired antibiotic resistance.

The bacterial strains that Applicants used are highly pathogenic to human beings. These strains include both gram-positive and gram-negative bacterial species.

All antibiotics used by Applicants were beta-lactams The beta-lactam antibiotics used are listed below with the date of their first medicinal use. The first three are classical penicillins while the others are other versions of beta-lactams.

Classical penicillins: Penicillin G (1941), ampicillin (1961), and amoxycillin (1972).

Other beta-lactams include: Cefoxitin, a cephamycin (1973) and penicillin G, a ureidopenicillin (1981).

The next three are third-generation cephalosporins include cefoperazone (1981), ceftriaxone (1982) and ceftazidime (1984).

These four antibiotics are more recent versions of beta-lactams: Aztreonam, a monobactam (1986), cefepime, a fourth-generation cephalosporin (1994), and meropenem, a carbapenem (1996).

Referring to FIG. 10, the illustrated chart summarizes test results based upon the use of a combination of 5-(Dihydroxyboryl)-2-thiophenecarboxylic acid with various antibiotics. The compound 5-(Dihydroxyboryl)-2-thiophenecarboxylic acid is a carboxylic acid derivative of thiophene. A dihydroxyboranyl group attached at the 5-position. As a boronic acid variant, the hydroxyl groups are attached to a boron atom. This compound has multiple applications including use as a chemical building block in the synthesis of various organic compounds. Such organic compounds include pharmaceuticals and dyes functioning as an intermediate capable of being modified and reacted further resulting in a broad range of molecules which demonstrate specific properties. The chemical formula for 5-(dihydroxyboryl)-2-thiophenecarboxylic acid is C5H5BO4S and its molecular weight is 171.97 g/mol.

As illustrated in FIG. 10, 5-(Dihydroxyboryl)-2-thiophenecarboxylic acid in combination with amoxycillin demonstrates enhanced effectiveness against Escherichia coli. The combination of 5-(Dihydroxyboryl)-2-thiophenecarboxylic acid and ampicillin demonstrates enhanced effectiveness against both MSSA (methicillin-sensitive Staphylococcus aureus) and MRCoNS (methicillin-resistant coagulase-negative Staphylococcus sps.). The combination of 5-(Dihydroxyboryl)-2-thiophenecarboxylic acid and ceftazidime demonstrates enhanced effectiveness against Escherichia coli, Klebsiella pneumoniae, Acinetobacter baumannii, and Citrobacter freundii. The combination of 5-(Dihydroxyboryl)-2-thiophenecarboxylic acid and ceftriaxone demonstrates enhanced effectiveness against Escherichia coli, Klebsiella pneumoniae, and Citrobacter freundii. The combination of 5-(Dihydroxyboryl)-2-thiophenecarboxylic acid and meropenem demonstrates enhanced effectiveness against Escherichia coli, Klebsiella pneumoniae, Acinetobacter baumannii, and Citrobacter freundii. The combination of 5-(Dihydroxyboryl)-2-thiophenecarboxylic acid and penicillin G demonstrates enhanced effectiveness against MSSA and MRCoNS. The combination of 5-(Dihydroxyboryl)-2-thiophenecarboxylic acid and aztreonam demonstrates enhanced effectiveness against Pseudomonas aeruginosa.

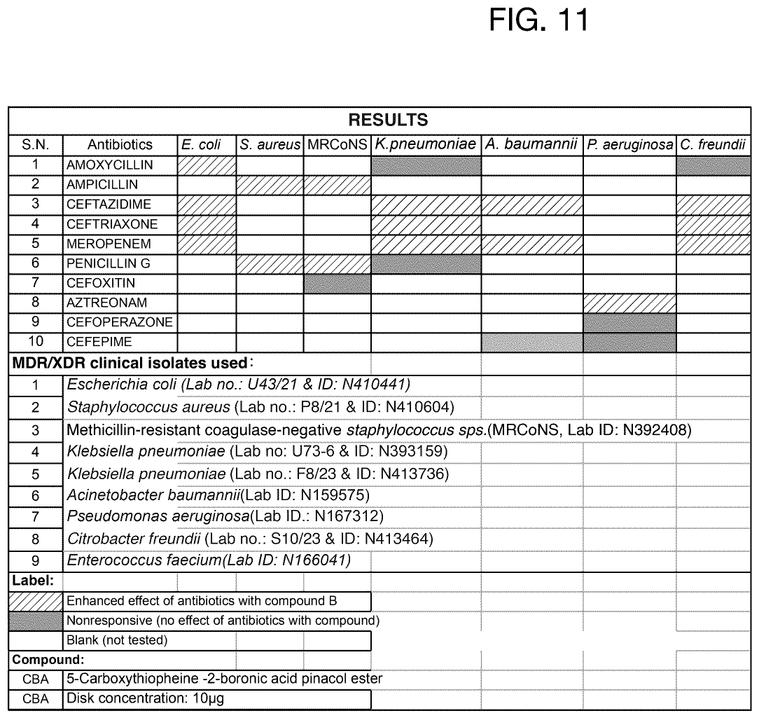

Referring to FIG. 11, the illustrated chart summarizes test results based upon the use of a combination of 5-Carboxythiophene-2-boronic acid pinacol ester with various antibiotics. The compound 5-Carboxythiophene-2-boronic acid pinacol ester is a solid having a melting point of between 176° C. and 181° C. 5-Carboxythiophene-2-boronic acid pinacol ester is a compound finding use in synthetic organic chemistry, particularly in Suzuki-Miyaura cross-coupling reactions. The compound also finds use in a variety of other metal-catalyzed processes. The chemical formula for 5-Carboxythiophene-2-boronic acid pinacol ester is C11H15BO4S. The molecular weight of 5-Carboxythiophene-2-boronic acid pinacol ester is 254.11 g/mol.

As illustrated in FIG. 11, 5-Carboxythiophene-2-boronic acid pinacol ester in combination with amoxycillin demonstrates enhanced effectiveness against Escherichia coli. The combination of 5-Carboxythiophene-2-boronic acid pinacol ester and ampicillin demonstrates enhanced effectiveness against both MSSA (methicillin-sensitive Staphylococcus aureus) and MRCoNS (methicillin-resistant coagulase-negative Staphylococcus sps.). The combination of 5-Carboxythiophene-2-boronic acid pinacol ester and ceftazidime demonstrates enhanced effectiveness against Escherichia coli, Klebsiella pneumoniae, Acinetobacter baumannii, and Citrobacter freundii. The combination of 5-Carboxythiophene-2-boronic acid pinacol ester and ceftriaxone demonstrates enhanced effectiveness against Escherichia coli, Klebsiella pneumoniae, and Citrobacter freundii. The combination of 5-Carboxythiophene-2-boronic acid pinacol ester and meropenem demonstrates enhanced effectiveness against Escherichia coli, Klebsiella pneumoniae, Acinetobacter baumannii, and Citrobacter freundii. The combination of 5-Carboxythiophene-2-boronic acid pinacol ester and penicillin G demonstrates enhanced effectiveness against MSSA and MRCoNS. The combination of 5-Carboxythiophene-2-boronic acid pinacol ester and aztreonam demonstrates enhanced effectiveness against Pseudomonas aeruginosa. The combination of 5-Carboxythiophene-2-boronic acid pinacol ester and cefoperazone also demonstrates enhanced effectiveness against Pseudomonas aeruginosa. The combination of 5-Carboxythiophene-2-boronic acid pinacol ester and cefepime demonstrates enhanced effectiveness against Acinetobacter baumannii and partial effectiveness against Pseudomonas aeruginosa.

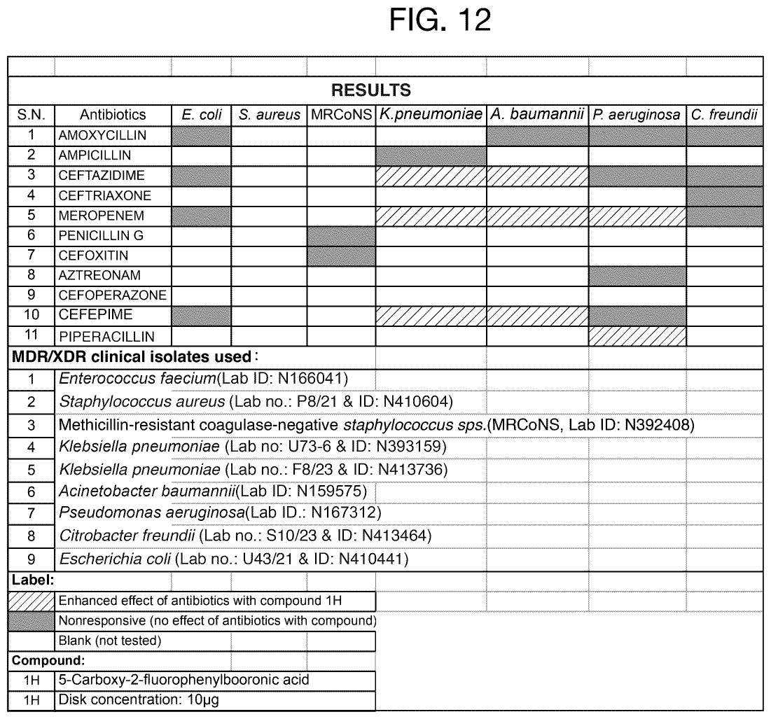

Referring to FIG. 12, the illustrated chart summarizes test results based upon the use of a combination of 5-Carboxy-2-fluorophenylboronic Acid with various antibiotics. The 5-Carboxy-2-fluorophenylboronic Acid is characterized by a benzene ring having a fluorine atom at position 2, a carboxyl group at position 5, and a boronic acid group, thus making it a derivative of both boronic and fluorbenzoic acid. It finds particular application in organometallic chemistry due to its novel reactivity as a boronic acid. Its electrophilic character is enhanced by the fluorine atom which allows it to form stable complexes with Lewis bases. The chemical formula for 5-Carboxy-2-fluorophenylboronic Acid is C7H6BFO4. The molecular weight of 5-Carboxy-2-fluorophenylboronic Acid is approximately 183.93 grams per mole.

As illustrated in FIG. 12, 5-Carboxy-2-fluorophenylboronic Acid in combination with ceftazidime demonstrates enhanced effectiveness against both Klebsiella pneumoniae and Acinetobacter baumannii. This compound also demonstrates enhanced effectiveness against Klebsiella pneumoniae, Acinetobacter baumannii and Pseudomonas aeruginosa in combination with meropenem. The same enhanced effectiveness against both Klebsiella pneumoniae and Acinetobacter baumannii was also observed when 5-Carboxy-2-fluorophenylboronic Acid was combined with cefepime. When combined with piperacillin, 5-Carboxy-2-fluorophenylboronic Acid proved effective against Pseudomonas aeruginosa.

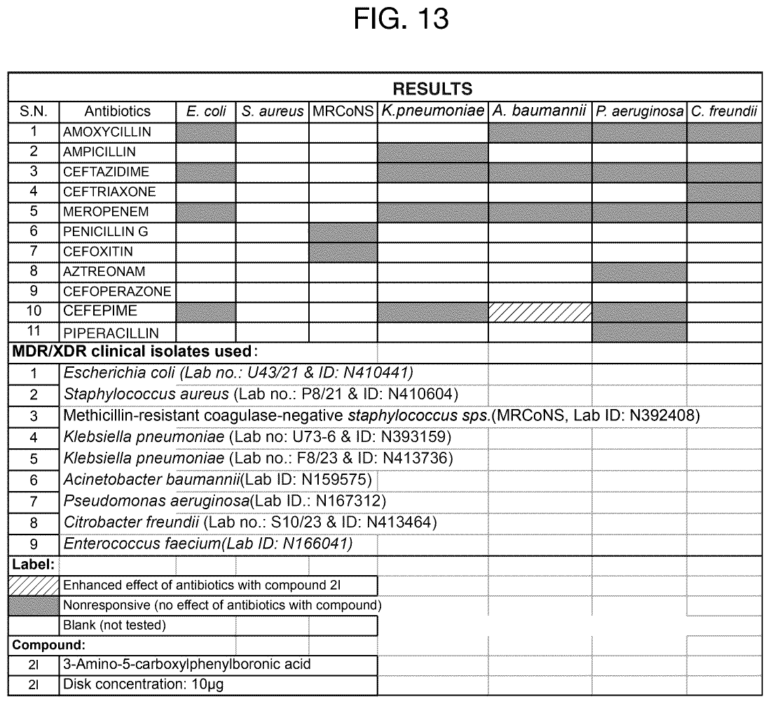

Referring to FIG. 13, the illustrated chart summarizes test results based upon the use of a combination of 3-Amino-5-carboxylphenylboronic acid with various antibiotics. A boron-containing organic acid, this has an amine group at the 3-position and a carboxyl group at the 5-position on a benzene ring. Because of this structure, the compound is occasionally referred to as 3-Amino-5-boronobenzoic acid. The compound finds use as a chemical intermediate in various chemical reactions. Some of these chemical reactions can be found in medicinal chemistry and the synthesis of a variety of molecules. The chemical formula for 3-Amino-5-carboxylphenylboronic acid is C7H8BNO4. The molecular weight of 3-Amino-5-carboxylphenylboronic acid is approximately 180.95 or 181.0 g/mol.

As illustrated in FIG. 13, 3-Amino-5-carboxylphenylboronic acid in combination with cefepime demonstrates enhanced effectiveness against Acinetobacter baumannii.

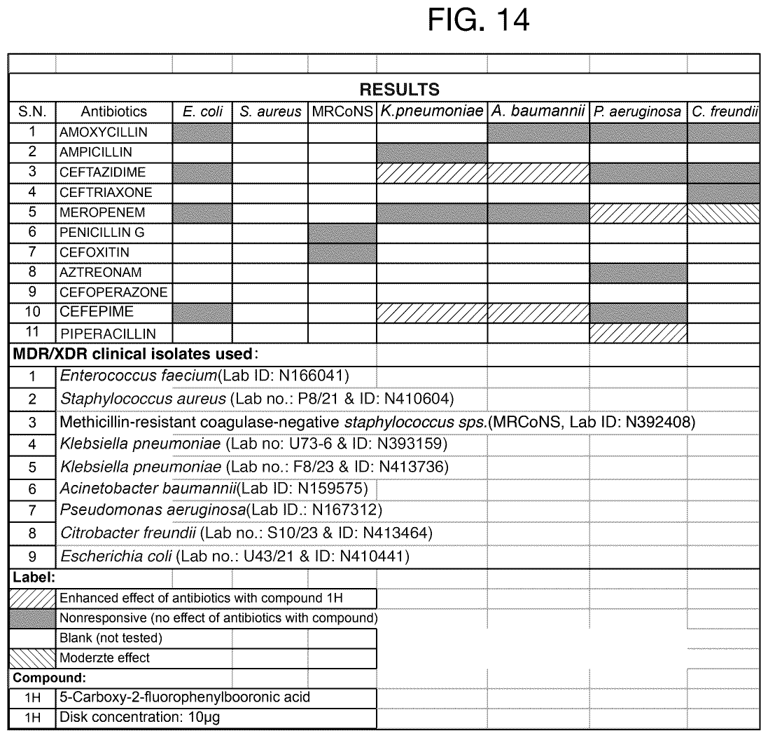

Referring to FIG. 14, the illustrated chart summarizes test results based upon the use of a combination of 5-Carboxy-2-chlorophenylboronic acid with various antibiotics. This compound often finds application as a reagent in organic synthesis and finds particular use in Suzuki-Miyaura coupling reactions. Used primarily as a reagent in organic synthesis, 5-Carboxy-2-chlorophenylboronic acid is oftentimes used in the development of pharmaceuticals and for sensor applications. It is highly valued in medicinal chemistry and material science given its ability to form carbon-carbon bonds. The chemical formula for 5-carboxy-2-chlorophenylboronic acid is C7H6BClO4. The molecular weight of 5-carboxy-2-chlorophenylboronic acid is 200.38 g/mol.

As illustrated in FIG. 14, 5-Carboxy-2-chlorophenylboronic acid in combination with ceftazidime demonstrates enhanced effectiveness against both Klebsiella pneumoniae and Acinetobacter baumannii. This compound also demonstrates enhanced effectiveness against Pseudomonas aeruginosa when combined with meropenem and piperacillin, while the same combination of meropenem demonstrates partial enhanced effectiveness against Citrobacter freundii. The same enhanced effectiveness against both Klebsiella pneumoniae and Acinetobacter baumannii was also observed when 5-Carboxy-2-chlorophenylboronic acid was combined with cefepime.

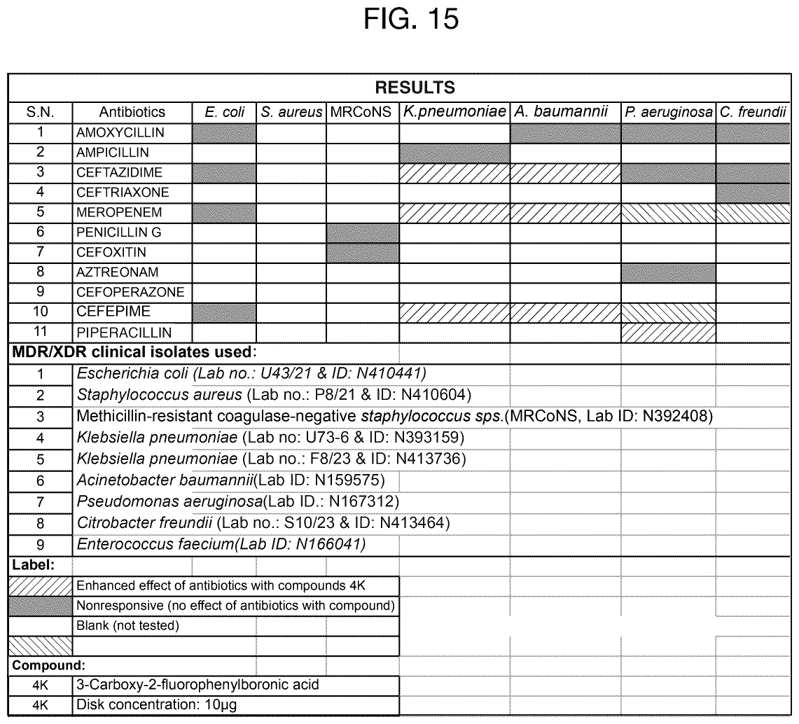

Referring to FIG. 15, the illustrated chart summarizes test results based upon the use of a combination of 3-Carboxy-2-fluorophenylboronic acid with various antibiotics. This compound has a phenyl ring. The compound is a fluorinated boronic acid having a carboxylic acid group in the 3-position and a fluorine atom in the 2-position of the phenyl ring. The compound is frequently used in proteomics research and finds particular application modifying proteins or peptides. It is primarily used as a building block in organic synthesis. Such uses are commonly directed to the development of pharmaceuticals, agrochemicals, and other advanced materials. The chemical formula for 3-Carboxy-2-fluorophenylboronic acid is C7H6BFO4. The molecular weight of 3-carboxy-2-fluorophenylboronic acid is 183.92954319999998.

As illustrated in FIG. 15, 3-Carboxy-2-fluorophenylboronic acid in combination with Ceftazidime demonstrates enhanced effectiveness against both Klebsiella pneumoniae and Acinetobacter baumannii. This compound also demonstrates enhanced effectiveness against both Klebsiella pneumoniae and Acinetobacter baumannii when combined with meropenem and demonstrates partial effectiveness against Pseudomonas aeruginosa and Citrobacter freundii. The same enhanced effectiveness against both Klebsiella pneumoniae and Acinetobacter baumannii was also observed when 3-Carboxy-2-fluorophenylboronic acid was combined with cefepime while this same combination demonstrates partial effectiveness against Pseudomonas aeruginosa. When 3-Carboxy-2-fluorophenylboronic acid was combined with piperacillin the combination also demonstrates partial effectiveness against Pseudomonas aeruginosa.

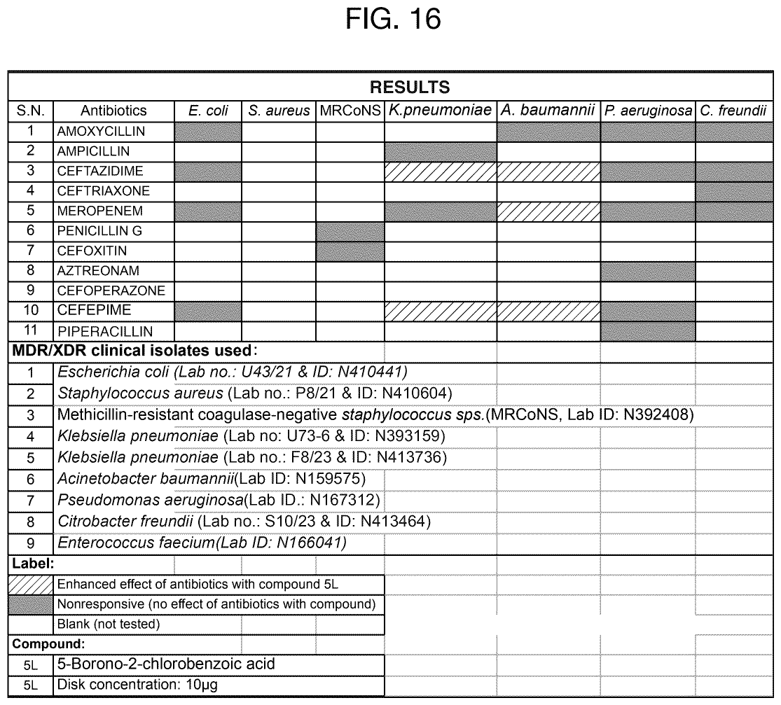

Referring to FIG. 16, the illustrated chart summarizes test results based upon the use of a combination of 5-Borono-2-chlorobenzoic acid with various antibiotics. This compound is a white solid and is primarily used as a precursor in the synthesis of various pharmaceutical compounds. Such compounds include nonsteroidal anti-inflammatory drugs, anticancer agents, and antipsychotics, and anticancer agents. The compound functions in certain chemical reactions by allowing for the modification and generation of more complex molecules having specific pharmacological properties. The chemical formula for 5-Borono-2-chlorobenzoic acid is C7H6BClO4. The molecular weight of 5-Borono-2-chlorobenzoic acid is approximately 200.4 g/mol.

As illustrated in FIG. 16, 5-Borono-2-chlorobenzoic acid in combination with ceftazidime demonstrates enhanced effectiveness against both Klebsiella pneumoniae and Acinetobacter baumannii. This compound also demonstrates enhanced effectiveness against Acinetobacter baumannii when combined with meropenem. The same enhanced effectiveness against both Klebsiella pneumoniae and Acinetobacter baumannii was also observed when 5-Borono-2-chlorobenzoic acid was combined with cefepime.

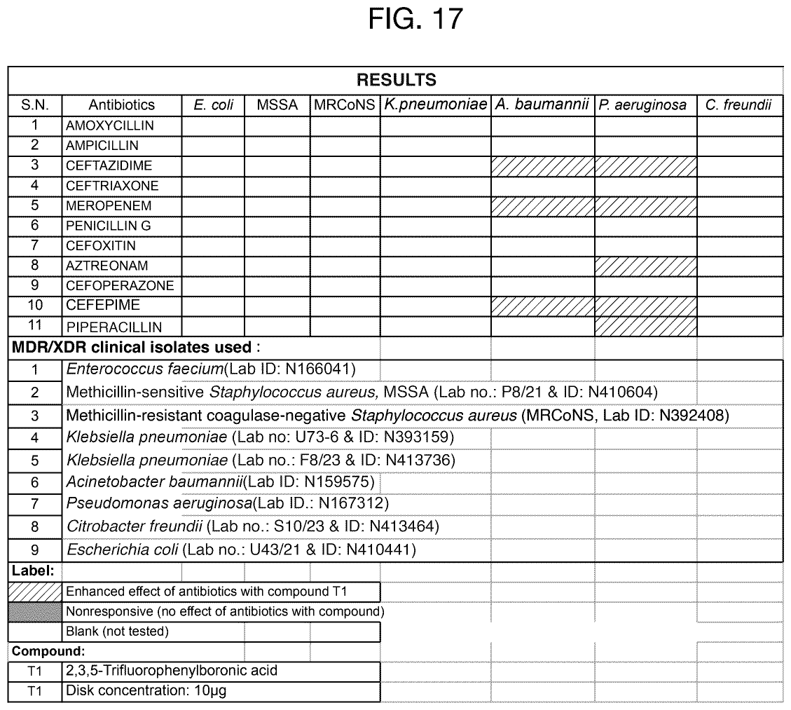

Referring to FIG. 17, the illustrated chart summarizes test results based upon the use of a combination of 2,3,5-Trifluorophenylboronic acid with various antibiotics. A boronic acid derivative having three fluorine atoms attached to a phenyl ring, 2,3,5-Trifluorophenylboronic acid is regarded as a versatile reagent which often finds application in organic synthesis. It is particularly useful in reactions such as Suzuki coupling in which carbon-carbon bonds are involved. The acid is often frequently used in the development of pharmaceuticals and other aspects of medicinal chemistry. The molecular weight of 2,3,5-trifluorophenylboronic acid is 175.90 g/mol. The chemical formula is C6H4BF3O2.

As illustrated in FIG. 17, 2,3,5-Trifluorophenylboronic acid in combination with ceftazidime demonstrates enhanced effectiveness against both Acinetobacter baumannii and Pseudomonas aeruginosa. Similarly, 2,3,5-Trifluorophenylboronic acid in combination with meropenem demonstrates enhanced effectiveness against both Acinetobacter baumannii and Pseudomonas aeruginosa. In combination with aztreonam, 2,3,5-Trifluorophenylboronic acid demonstrates enhanced effectiveness against Pseudomonas aeruginosa. In combination with cefepime, 2,3,5-Trifluorophenylboronic acid demonstrates enhanced effectiveness against both Acinetobacter baumannii and Pseudomonas aeruginosa. And in combination with piperacillin, 2,3,5-Trifluorophenylboronic acid demonstrates enhanced effectiveness against Pseudomonas aeruginosa.

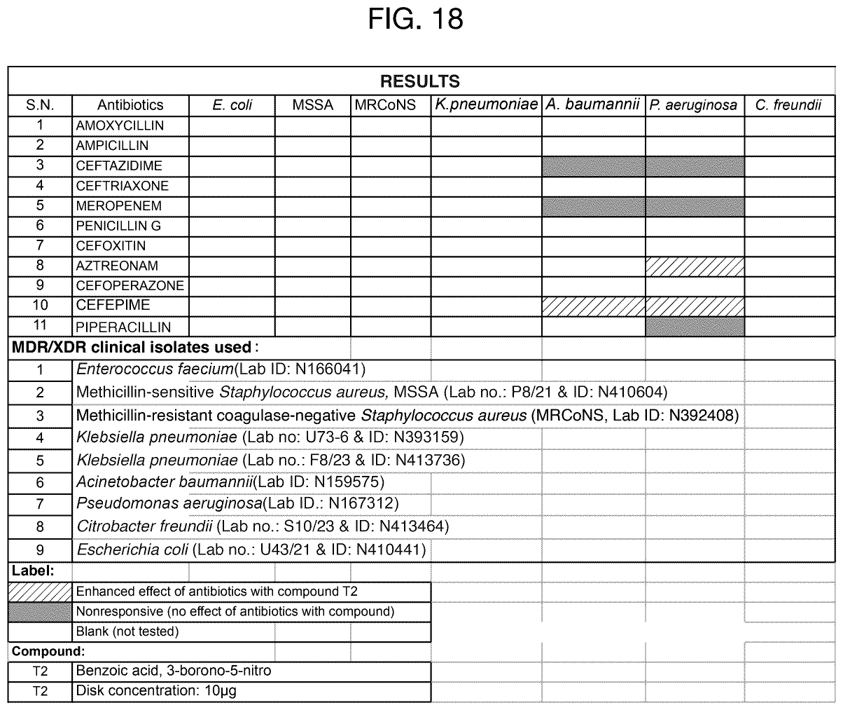

Referring to FIG. 18, the illustrated chart summarizes test results based upon the use of a combination of Benzoic acid, 3-borono-5-nitro with various antibiotics. A boronic acid, Benzoic acid, 3-borono-5-nitro is a derivatized benzoic acid with a boronic acid group and a nitro group positioned respectively at the 3- and 5-positions, respectively. Also known as 3-carboxy-5-nitrophenylboronic acid, this compound is frequently used as a reactant in organic synthesis and, as such, is often used in Suzuki-Miyaura cross-coupling reactions. The molecular weight of Benzoic acid, 3-borono-5-nitro is 210.94 g/mol and has a chemical formula of C7H6BNO6.

As illustrated in FIG. 18, Benzoic acid, 3-borono-5-nitro in combination with aztreonam, demonstrates enhanced effectiveness against Pseudomonas aeruginosa. In combination with cefepime, Benzoic acid, 3-borono-5-nitro demonstrates enhanced effectiveness against both Acinetobacter baumannii and Pseudomonas aeruginosa.

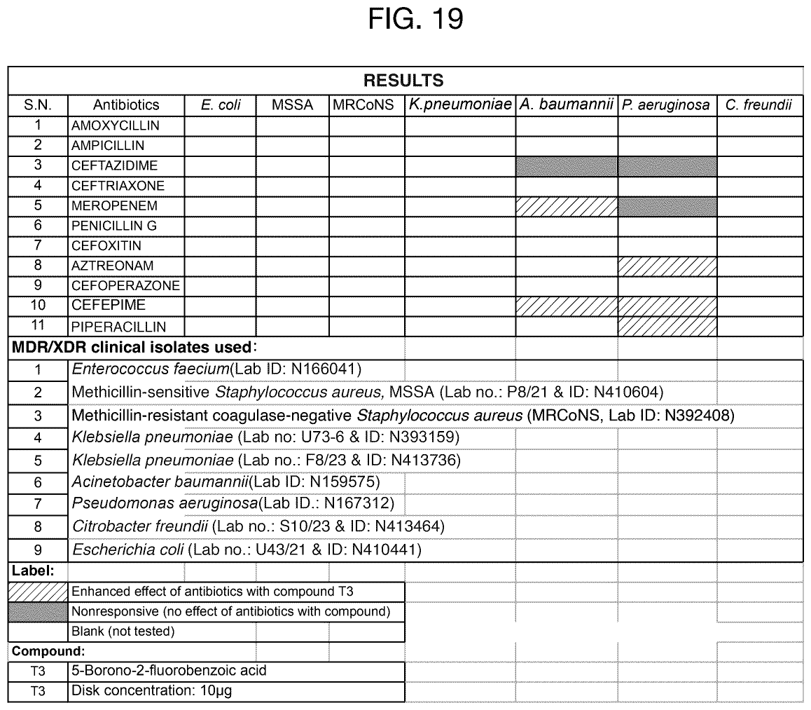

Referring to FIG. 19, the illustrated chart summarizes test results based upon the use of a combination of 5-Borono-2-fluorobenzoic acid with various antibiotics. A white solid, 5-Borono-2-fluorobenzoic acid is used most commonly as a precursor in the synthesis of various pharmaceuticals and organic compounds. It is a derivative of benzoic acid having a fluorine atom at the 2-position and a boron atom at the 5-position. The combination also finds application in reactions involving boron-containing molecules. The compound also has been studied to determine is potential as a chiral auxiliary in organic synthesis. It is known that 5-Borono-2-fluorobenzoic acid is a building block in the development of new pharmaceuticals containing boron. The molecular weight of 5-Borono-2-fluorobenzoic acid is 183.930 g/mol and has a chemical formula of C7H6BFO4.

As illustrated in FIG. 19, 5-Borono-2-fluorobenzoic acid in combination with meropenem demonstrates enhanced effectiveness against Acinetobacter baumannii. In combination with aztreonam, 5-Borono-2-fluorobenzoic acid demonstrates enhanced effectiveness against Pseudomonas aeruginosa. In combination with cefepime, 5-Borono-2-fluorobenzoic acid demonstrates enhanced effectiveness against both Acinetobacter baumannii and Pseudomonas aeruginosa. And in combination with piperacillin, 5-Borono-2-fluorobenzoic acid demonstrates enhanced effectiveness against Pseudomonas aeruginosa.

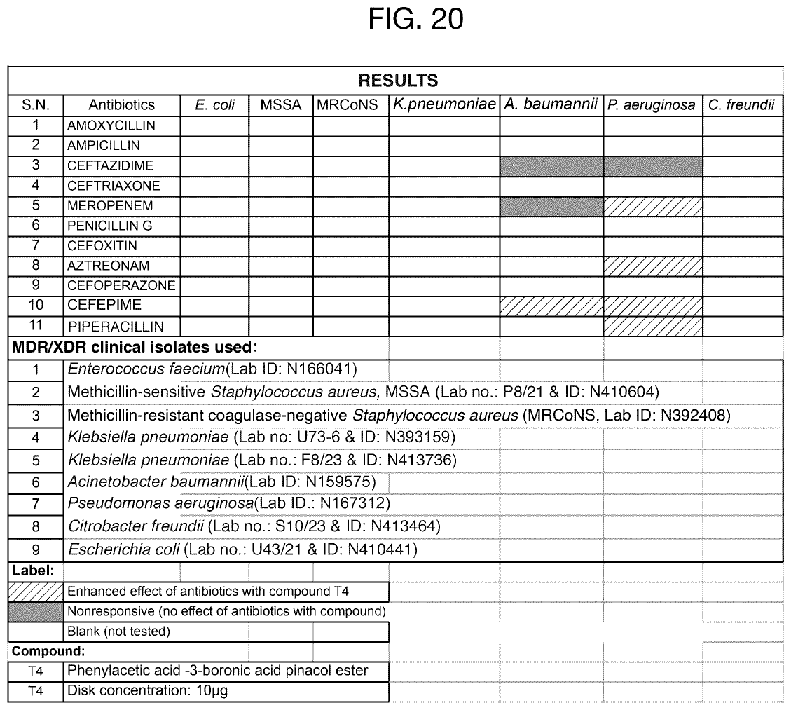

Referring to FIG. 20, the illustrated chart summarizes test results based upon the use of a combination of Phenylacetic acid-3-boronic acid pinacol ester with various antibiotics. Phenylacetic acid-3-boronic acid pinacol ester is an organic building block and functions as a fundamental component in the synthesis of complex organic molecules. Accordingly, Phenylacetic acid-3-boronic acid pinacol ester serves as a basic unit from which larger and more intricate structures are constructed. These versatile compounds are essential in various chemical compositions including pharmaceutical compositions. The molecular weight of Phenylacetic acid-3-boronic acid pinacol ester is 262.11 g/mol. It has a chemical formula of C14H19BO4.

As illustrated in FIG. 20, Phenylacetic acid-3-boronic acid pinacol ester in combination with meropenem demonstrates enhanced effectiveness against Pseudomonas aeruginosa. In combination with aztreonam, Phenylacetic acid-3-boronic acid pinacol ester demonstrates enhanced effectiveness against Pseudomonas aeruginosa. In combination with cefepime, Phenylacetic acid-3-boronic acid pinacol ester demonstrates enhanced effectiveness against both Acinetobacter baumannii and Pseudomonas aeruginosa. And in combination with piperacillin, Phenylacetic acid-3-boronic acid pinacol ester demonstrates enhanced effectiveness against Pseudomonas aeruginosa.

In addition to the above discoveries, the inventors herein identified two likely variations in the boronic acid group. One of these variations involves the use of a borinic acid group, with one alkyl group (methyl, ethyl, or more) and one OH group. The other of these variations involves the use of a boronic ester having one or both OH groups esterified to an alcohol, in either a cyclic form or noncyclic form. The inventors herein used pinanediol, which has both boronic acid OH groups esterified. But having only one esterified boronic acid OH group is possible.

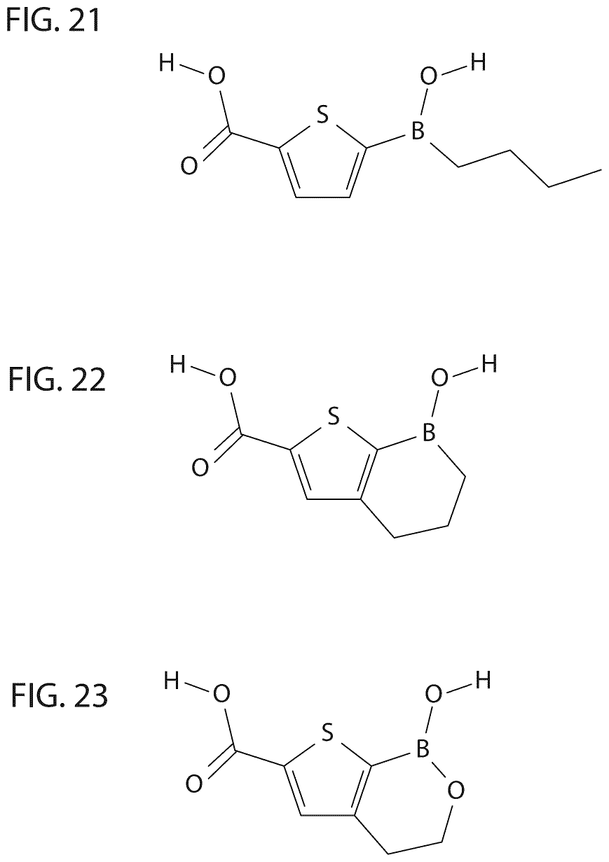

Such modifications are illustrated in FIGS. 21-23. The carboxylic acid would, of course, be in the ionized, anionic form in solution.

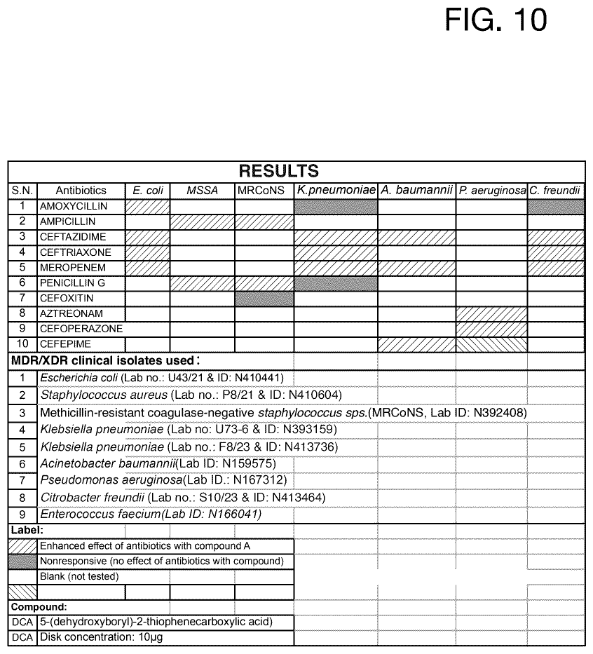

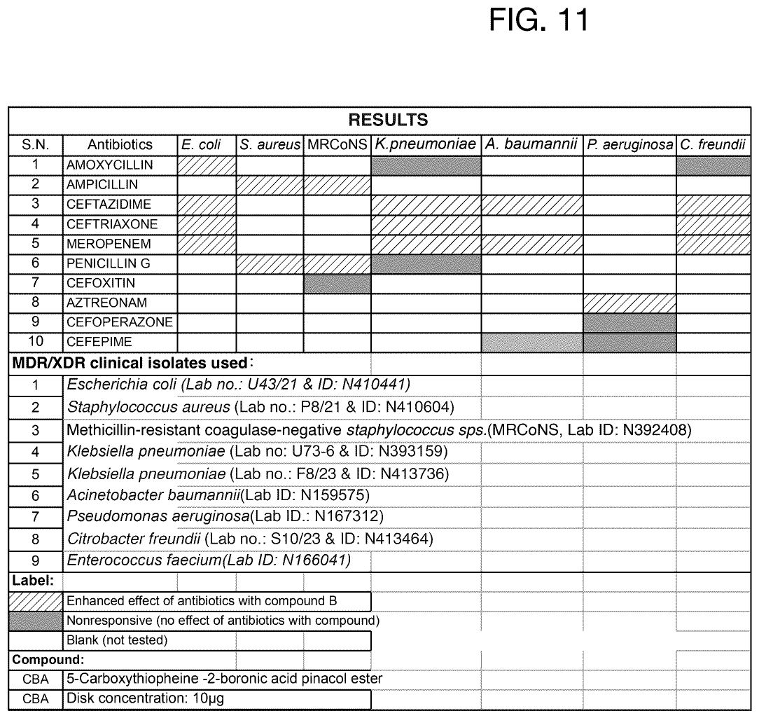

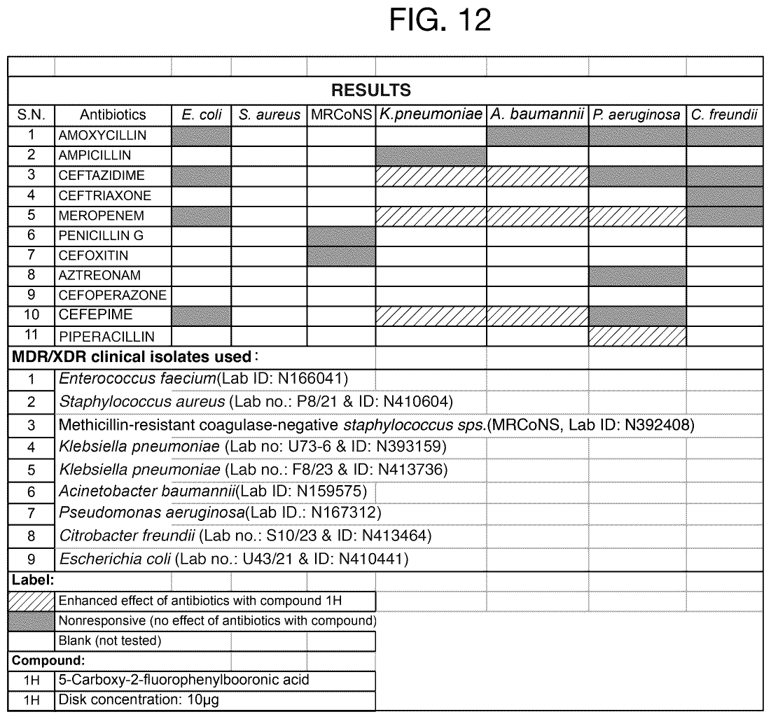

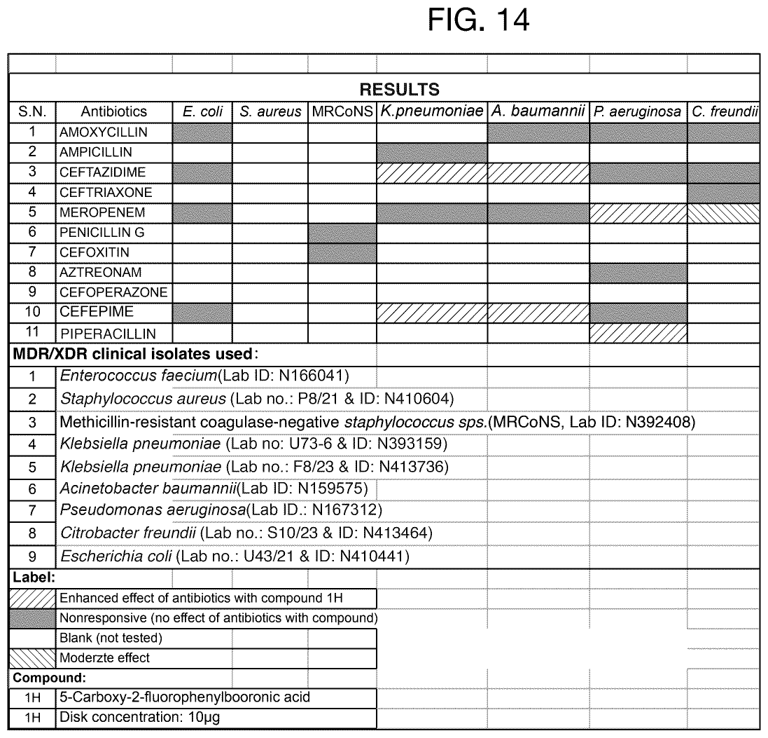

Referring to FIG. 21, an exemplary boronic acid having a n-butyl group on the boron atom is illustrated. In FIG. 22, a boronic acid of FIG. 21 is illustrated in cyclic form. In FIG. 23, a cyclic boronic acid ester is illustrated.

### Example

According to the disclosed invention, requisite amounts of both cefepime and 5-Boronothiophene-2-carboxylic acid are dissolved. Particularly, 2.0 g of cefepime are dissolved in sterile normal saline (0.9% sodium chloride) solution and 0.5 g of 5-Boronothiophene-2-carboxylic acid are dissolved in DMSO. Dissolution may be accelerated by use of an alcohol such as ethyl alcohol. Thereafter, a 2.5 g dose of a combination of cefepime (2.0 g) and 5-Boronothiophene-2-carboxylic acid (0.5 g) together with sterile normal saline (0.9% sodium chloride) as a diluent is administered to randomly assigned adult patients challenged with complicated urinary tract infection, including acute pyelonephritis, over a two-hour period with the dose being administered intravenously every 8 hours. A 1.0 g dose of a placebo is administered over 30-minute periods, every 8 hours also intravenously. The administration may be carried out over a period of 7 days. The anticipated results demonstrate the patients experienced both microbiologic and clinical success.

The conjunction “or” is inclusive. The terms “first”, “second”, “third”, etc. are used to distinguish respective elements and are not used to denote a particular order of those elements unless otherwise specified or order is explicitly described or required.

Numerous specific details are set forth to provide a thorough understanding of the claimed subject matter. However, those skilled in the art will understand that the claimed subject matter may be practiced without these specific details. In other instances, methods, apparatuses or systems that would be known by one of ordinary skill have not been described in detail so as not to obscure claimed subject matter.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, it should be understood that the present disclosure has been presented for purposes of example rather than limitation, and does not preclude inclusion of such modifications, variations and/or additions to the present subject matter as would be readily apparent to one of ordinary skill in the art.

We claim:

1. A composition for the treatment of disease comprising one or more of 5-Carboxythiophene-2-boronic acid pinacol ester, 5-Carboxy-2-fluorophenylboronic Acid, 3-Amino-5-carboxylphenylboronic acid, 5-Carboxy-2-chlorophenylboronic acid, 3-Carboxy-2-fluorophenylboronic acid, 5-dehydroxyboryl-2-thiophenecarboxylic acid, and 5-Borono-2-chlorobenzoic acid in combination with an antibiotic.

2. The composition of claim 1 wherein the antibiotic is selected from the group consisting of amoxycillin, ampicillin, ceftazidime, ceftriaxone, meropenem, penicillin G, cefoxitin, aztreonam, cefoperazone, and cefepime.

3. An antibiotic composition comprising:
a beta-lactam antibiotic, and
a beta-lactamase inhibitor, the beta-lactamase inhibitor being selected from the group consisting of boronic-acid beta-lactamase inhibitors, boronic-ester beta-lactamase inhibitors, carboxyl-group beta-lactamase inhibitors, halogen-terminated heterocyclic group beta-lactamase inhibitors, and pharmaceutically acceptable salts thereof.

4. The antibiotic composition according to claim 3, wherein the beta-lactamase inhibitor comprises a carboxyl group and a boronic-acid or boronic-ester group.

5. The antibiotic composition according to claim 4, wherein the beta-lactamase inhibitor comprises a heterocyclic ring.

6. The antibiotic composition according to claim 5, wherein the beta-lactamase inhibitor comprises one or more atoms selected from the group consisting of S, Se, B, and O as members of the heterocyclic ring.

7. The antibiotic composition according to claim 6, wherein the beta-lactamase inhibitor comprises a ring structure comprising five atoms.

8. The antibiotic composition according to claim 3, wherein the beta-lactamase inhibitor comprises a halogen group.

9. The antibiotic composition according to claim 3, wherein the beta-lactamase inhibitor comprises at least one boronic-acid group.

10. The antibiotic composition according to claim 9, wherein the beta-lactamase inhibitor comprises at least two boronic-acid groups.

11. The antibiotic composition according to claim 10, wherein the molar ratio of the beta-lactamase inhibitor to the beta-lactam antibiotic is at least 1.

12. The antibiotic composition according to claim 11, wherein the molar ratio of the beta-lactamase inhibitor to the beta-lactam antibiotic is at least 5.

13. The antibiotic composition according to claim 12, wherein the molar ratio of the beta-lactamase inhibitor to the beta-lactam antibiotic is at least 10.

14. A method of treating a bacterial infection using the composition according to claim 3, the method comprising administering the composition of claim 3 to a location of bacterial infection, the bacterial infection comprising bacteria that are resistant to the beta-lactam antibiotic.

15. The method of claim 14, wherein the beta-lactamase inhibitor alone without the beta-lactam antibiotic does not provide anti-microbial activity against the bacterial infection.

16. The method of claim 15, wherein the method comprises administering the composition to a human or an animal.

17. A method of treating a bacterial infection, the method comprising:
administering a beta-lactam antibiotic to the bacterial infection; and
administering a beta-lactamase inhibitor to the bacterial infection,
wherein the bacterial infection comprises bacteria that are resistant to the beta-lactam antibiotic if the beta-lactam antibiotic is administered to the bacterial infection without the presence of the beta-lactamase inhibitor.

18. The method of claim 17, wherein the beta-lactamase inhibitor does not provide significant antimicrobial activity against the bacterial infection when administered to the bacterial infection without the beta-lactam antibiotic and wherein the beta-lactam antibiotic is a beta-lactam in the penicillin and cephalosporin families.

19. The method of one of claim 18, wherein the beta-lactamase inhibitor is selected from the group consisting of a carboxyl group, boronic-acid beta-lactamase inhibitors, boronic-ester beta-lactamase inhibitors, and pharmaceutically acceptable salts thereof.

20. The method of claim 18, wherein the beta-lactamase inhibitor is selected from the group consisting of a five-member heterocyclic compound, 2-Carboxythiophene-5-boronic Acid or an ester or pharmaceutically acceptable salt thereof.
